(19) United States
(12) Patent Application Publication
(10) Pub. No.: US 2010/0256484 A1
(43) Pub. Date: Oct. 7, 2010

(54) OPTICALLY MATCHING MEDIUM AND METHOD FOR OBTAINING SUCH A MEDIUM

(75) Inventors: Antonius Wilhelmus Maria De Laat, Eindhoven (NL); Martinus Bernardus Van Der Mark, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/740,141

(22) PCT Filed: Oct. 28, 2008
(86) PCT No.: PCT/IB2008/054436

§ 371 Date: Apr. 28, 2010

(30) Foreign Application Priority Data
Nov. 5, 2007 (EP) 07119950.9

(51) Int. Cl. A61B 5/00; A61K 49/04

(52) U.S. Cl. 600/425; 424/9.4; 600/476

(57) ABSTRACT

An optically matching medium (6) for use in a device for examining turbid media is provided. The optically matching medium (6) comprises an absorber component (8) providing the optically matching medium with predetermined absorption properties. The absorber component (8) is selected such that the optical properties of the optically matching medium (6), with respect to electromagnetic radiation having a wavelength between 650 nm and 900 nm, substantially match the optical properties of a turbid medium (1) to be examined. The absorber component (8) comprises a dye or pigment approved for use in cosmetics, food, and/or drugs.

### FIELD OF INVENTION

The present invention relates to an optically matching medium and to a method for obtaining an optically matching medium. In particular, the present invention relates to an optically matching medium for use in a device for examining turbid media.

Moreover, the present invention relates to a device for examining turbid media and in particular to a medical image acquisition device.

### BACKGROUND OF THE INVENTION

In the context of the present application, the term turbid medium is to be understood to mean a substance consisting of a material having a high light scattering coefficient, such as for example an Intralipid solution or biological tissue. Further, light is to be understood to mean electromagnetic radiation of a wavelength in the range from 400 nm to 1400 nm. The term “optical properties” covers the reduced scattering coefficient μ′ₛ and the absorption coefficient μₐ. Furthermore, “matching optical properties” is to be understood as having a similar reduced scattering coefficient μ′ₛ and a similar absorption coefficient μₐ.

In recent years, several methods and devices for examining turbid media, e.g. female breast tissue, have been developed. In particular, new devices for detection and analysis of breast cancer have been developed and existing technologies have been improved.

WO 00/56206 A1 discloses a device for imaging the interior of a turbid medium by using light sources to irradiate the turbid medium and photodetectors for measuring a part of the light transported through the turbid medium. A control unit is provided for reconstructing an interior of the turbid medium on the basis of the measured intensities. The disclosed device is particularly adapted for examining female breasts. In order to allow the examination of the turbid medium, the device is provided with a holder enclosing a measuring volume and arranged to receive the turbid medium. The light used for examining the turbid medium has to be transmitted from the light sources to the turbid medium and from the turbid medium to the photodetectors. Due to different sizes of the turbid media to be examined, the size of the holder for receiving the turbid medium does not perfectly match the size of the turbid medium, i.e. a space remains between the holder and the turbid medium. A number of light sources and a number of photodetectors are distributed across the wall of the holder. The space between the holder and the turbid medium is filled with a so-called optically matching fluid as an optically matching medium. The optically matching fluid provides optical coupling between the part of the turbid medium to be imaged and the light sources and the photodetectors, respectively. A matching fluid is used the optical properties of which substantially match the optical properties of the turbid medium to be examined. The optically matching fluid is intended to prevent optical short-cutting between the light sources and the photodetectors, i.e. light transmitted from the light sources to the photodetectors without being transmitted through the turbid medium. The optically matching fluid counteracts boundary effects in the reconstructed image which are caused by the difference in optical contrast between the interior of the turbid medium in the holder and the remaining space in the holder. In the disclosed device, the light sources alternately irradiate the turbid medium and the photodetectors measure a part of the light transmitted through the turbid medium. A plurality of such measurements are performed and, based on the results of the measurements, the control unit reconstructs the image of the examined turbid medium.

Several types of devices for imaging the interior of a turbid medium by use of light have been developed. Examples for such devices are mammography devices and devices for examining other parts of human or animal bodies. A prominent example for a method for imaging the interior of a turbid medium is Diffuse Optical Tomography (DOT). In particular, such devices are intended for the localization of inhomogeneities in in vivo breast tissue of a part of a breast of a female human body. A malignant tumor is an example for such an inhomogeneity. The devices are intended to detect such inhomogeneities when they are still small, so that for example carcinoma can be detected at an early stage. A particular advantage of such devices is that the patient does not have to be exposed to the risks of examination by means of ionizing radiation, as e.g. X-rays.

In recent years, new approaches for further enhancing methods for detecting breast cancer by use of light have been made. For example, a fluorescent dye has been developed which can be injected into the body and will accumulate in cancer cells. If this fluorescent dye then becomes excited with light of a suitable wavelength, the locally emitted light can be detected. Based on the emitted light size and localization of carcinoma can be determined. Thus a powerful method for detection and localization of breast cancer is provided. It has been found that light for excitation having specific wavelengths is required in these new developed techniques. In particular, light having a wavelength in the range from 650 nm to 900 nm provides promising results. In recent experiments a laser having a wavelength of 730 nm has been used as a light source. However, the known optically matching media have the disadvantage that no or only poor absorption is provided with respect to wavelengths above 700 nm. Thus, the known optically matching media are not well suited for use in the new techniques. As a consequence, a need to develop a new optically matching medium suitable for use with a fluorescent dye as a contrast agent has arisen. A particular problem arises in that the new optically matching medium has to be suitable for direct contact to the turbid medium to be examined.

Optically matching media suitable for this purpose must provide optical properties, in particular a reduced scattering coefficient μ′ₛ and an absorption coefficient μₐ, matching the optical properties of the turbid medium to be examined in the desired range of wavelengths. Further, it is necessary that the optically matching medium can be used in direct contact with the turbid medium to be examined without being harmful. This is particularly relevant for use in contact with in vivo human tissue. Furthermore, the possibility of a cost-efficient production of the optically matching medium is required. In particular, for applications with different types of turbid media and for applications requiring a new range of wavelengths, a fast adaptation of the properties of an optically matching medium at low costs would be advantageous.

### SUMMARY OF THE INVENTION

It is an object of the present invention to provide an optically matching medium with appropriate optical properties in the desired range of wavelengths which can be used in direct contact with the turbid medium and which can be produced in a cost-efficient way.

This object is solved by the optically matching medium as defined in claim 1.

The optically matching medium for use in a device for examining turbid media comprises an absorber component providing the optically matching medium with predetermined absorption properties. The absorber component is selected such that the optical properties of the optically matching medium, with respect to electromagnetic radiation having a wavelength between 650 nm and 900 nm, substantially match the optical properties of the turbid medium to be examined. The absorber component comprises a dye or pigment approved for use in cosmetics, food, and/or drugs.

Since the optical properties of the optically matching medium match the optical properties of the turbid medium with respect to the wavelength between 650 nm and 900 nm, optical boundary effects can be reliably reduced in the device for examining the interior of turbid media even in the case that a fluorescent dye is used as a contrast agent. Since the absorber component comprises a dye or pigment which is approved for use in cosmetics, food, and/or drugs, the desired optical properties can be provided to the optically matching medium in a cost-efficient way and it can be reliably ensured that the contact to the turbid medium does not cause any problems.

Preferably, the absorber component comprises a combination of at least two dyes or types of pigments approved for use in cosmetics, food, and/or drugs. According to this feature, the optical properties of the optically matching medium can be accurately designed. Two dyes or types of pigments having different optical properties can be combined to achieve an optically matching medium the optical properties of which are similar to the turbid medium. Further, contact of the optically matching medium to the turbid medium will not cause any problems.

If the absorber comprises a dye or a combination of dyes from the group having the Color Index (CI): 74220; 74160; 77266; 74260; 61570; 75815; and 10020, the optically matching medium can be produced by using commercially available dyes. Thus, it can be produced in a cost-efficient way. Further, it has been found that these dyes are well suited in order to achieve matching optical properties.

According to an aspect, the dye or type of pigments is selected such that the optical properties of the optically matching medium match the optical properties of the turbid medium with respect to electromagnetic radiation having a wavelength in a range around 730 nm, preferably between 700 nm and 800 nm, more preferably between 650 nm and 900 nm. If the dye or type of pigments is selected in this way, an optically matching medium can be provided which is particularly well suited for the application in Diffuse Optical Tomography (DOT) detecting fluorescent dye accumulated in cancer cells.

Preferably, the optically matching medium further comprises a scattering component which provides predetermined scattering properties to the optically matching medium. By providing the scattering component, the optically matching medium can reliably prevent optical short-cutting in the device for examining the interior of turbid media. Further, the scattering properties of the optically matching medium can be adapted to the scattering properties of the turbid medium to be examined. If the scattering component comprises titanium dioxide of a type released for cosmetics, food, and/or drugs, the effect can be achieved in a particularly effective way and it is ensured that the optically matching medium is suitable for direct contact to the turbid medium.

Preferably, the optically matching medium is adapted to be placed between a turbid medium to be examined and a holder being a part of the device for examining turbid media which is structured to receive the turbid medium to be examined. More preferably, the optically matching medium is adapted for use in a device for optical mammography, in particular for use in a device for Diffuse Optical Tomography (DOT), and the optical properties of the optically matching medium are provided to substantially match the optical properties of a female breast as a turbid medium. Thus, the optical properties of the optically matching medium are specifically adapted to the desired application.

The object is also solved by a method for obtaining an optically matching medium for use in a device for examining turbid media according to claim 9. At least one appropriate dye or type of pigments is selected based on the optical properties of a turbid medium to be examined. The optically matching medium is generated by adding the at least one selected dye or type of pigments as an absorber component to a fluid. The absorber component is selected such that, after adding the absorber component to the fluid, the optical properties of the optically matching medium substantially match the optical properties of a turbid medium to be examined with respect to electromagnetic radiation having a wavelength between 650 nm and 900 nm. In the step of selecting at least one dye or type of pigments, a dye or type of pigments is selected which is approved for use in cosmetics, foods and/or drugs.

By selecting the absorber component with optical properties matching the optical properties of the turbid medium with respect to the wavelength between 650 nm and 900 nm, optical boundary effects can be reduced in the device for examining the interior of turbid media even for the particular case that a fluorescent dye is used as a contrast agent. Since the absorber component comprises a dye or pigment which is approved for use in cosmetics, food, and/or drugs, the desired optical properties can be provided to the optically matching medium in a cost-efficient way and it can be reliably ensured that the contact to the turbid medium is not harmful. Thus, no elaborated tests for establishing approval for medical applications are necessary.

Further, a device for examining turbid media is provided. The device comprises a holder for receiving a turbid medium to be examined; at least one light source optically connected to the holder for irradiating the interior of the holder; at least one detector optically connected to the holder for detecting light emanating from the interior of the holder; and an optically matching medium according to any one of claims 1 to 8. This device achieves the advantages described above with respect to the optically matching medium. Preferably, the device is a medical image acquisition device,

The described advantages can also be achieved by the disclosed use of an optically matching medium in a device for examining turbid media. The device for examining turbid media comprises a holder for receiving a turbid medium to be examined; at least one light source optically connected to the holder for irradiating the interior of the holder; and at least one detector optically connected to the holder for detecting light emanating from the interior of the holder. The optically matching medium is an optically matching medium according to any one of claims 1 to 8 and is arranged between the holder and the turbid medium.

The medical image acquisition device also achieves the advantages described with respect to the device.

### BRIEF DESCRIPTION OF THE DRAWINGS

Further features and advantages of the present invention will arise from the description of embodiments with reference to the enclosed drawings.

FIG. 1 is a schematic illustration of a part of a device for examining turbid media.

FIG. 2 schematically shows the process of generating an optically matching medium.

### DETAILED DESCRIPTION OF EMBODIMENTS

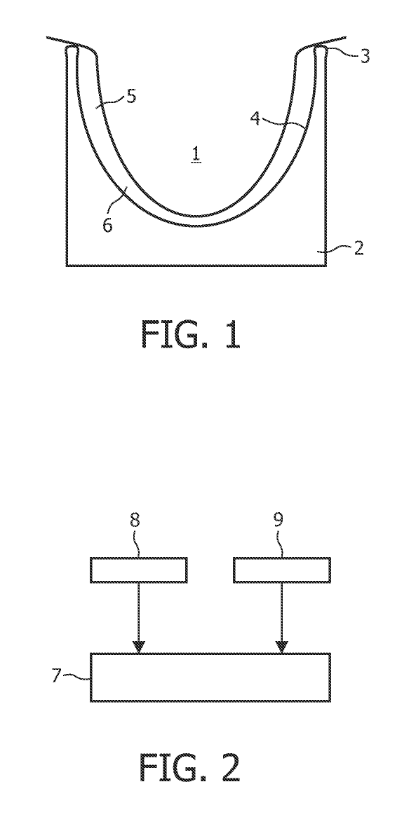

An embodiment of the present invention will be described with reference to FIG. 1. In FIG. 1, a part of a device for examining turbid media is shown. In the embodiment, the device for examining turbid media is realized as an apparatus for Diffuse Optical Tomography (DOT) for in vivo examination of biological tissue. In particular, the device is adapted for examining a female human breast 1 as a turbid medium. The device for examining turbid media comprises a holder 2 which is adapted for receiving the breast 1. The holder 2 has a cup-like shape which is closed at the bottom and has an opening at the upper side. The opening is surrounded by a rim 3 and the interior of the holder 2 is formed as a cavity. For examination, the breast 1 to be examined is placed in the holder 2 such that it freely hangs in the holder 2 and the tissue surrounding the breast 1 rests on the rim 3, as can be seen in FIG. 1. The inner surface 4 of the holder 2 is provided with a plurality of fibers (not shown) illuminating the breast 1 with light from a laser as a light source and transmitting light from the breast 1 to detectors. The detailed construction of the device for examining turbid media will not be described, since it is known to the person skilled in the art. In the present embodiment the device for examining turbid media is in principle realized as an apparatus disclosed in the prior art WO 00/56206 A1 which has been described in the introductory part of the specification. However, the device for examining turbid media is adapted to work with lasers emitting light having wavelengths in the range from approximately 650 nm to approximately 900 nm. In a particular embodiment, the device is adapted to work with laser light having a wavelength of 730 nm.

As can be seen in FIG. 1, the size of the holder 2 is such that a space 5 remains between the inner surface 4 and the breast 1 placed in the holder 2. For examination, the space 5 is filled with an optically matching medium 6 which serves to provide optical coupling between the breast 1 to be imaged and the fibers coupling to the light source and detectors, respectively. The optically matching medium 6 further serves to prevent optical short-cutting between the fibers coupling to the light source and the fibers coupling to the photodetectors. Furthermore, the optically matching medium 6 serves to counteract boundary effects in the reconstructed image which are caused by the difference in optical contrast between the interior of the breast 1 in the holder 2 and the remaining space 5 in the holder 2.

According to the present embodiment, the optically matching medium 6 is specifically formulated for the application in Diffuse Optical Tomography (DOT). The optically matching medium 6 is specifically formulated to provide the required optical properties with respect to light having a wavelength in the range from 650 nm to 900 nm. More specifically, the optically matching medium is formulated to provide the required optical properties in a range around the wavelength which is used in the specific device for examining turbid media, e.g. 730 nm.

Next, it will be described which optical properties are required for the optically matching medium. In order to achieve the best possible results, the optical absorption and scattering of the optically matching medium 6 should mimic the optical properties of the turbid medium to be examined which in the present embodiment is the breast 1. Thus, the optical properties of the optically matching medium 6 are adjusted such that the reduced scattering coefficient μ′ₛ and the absorption coefficient μₐ are substantially similar to the reduced scattering coefficient and the absorption coefficient of the turbid medium with respect to the range of wavelengths relevant for the device for examining turbid media.

With respect to FIG. 2 it will be described how the appropriate optically matching medium is composed.

The optically matching medium 6 comprises a base medium 7 which might be e.g. water or another medium suitable for direct contact to the turbid medium. Other suitable base media are solvents used in cosmetics, such as e.g. propylene glycol, glycerol, or mixtures of these with water. In order to achieve the desired absorption coefficient μₐ, an absorber component 8 is added which comprises the appropriate absorption properties. In order to comply with the requirement that the optically matching medium 6 has to be suitable for direct contact to the turbid medium, an appropriate absorber component 8 has to be chosen. According to the present embodiment, the absorber component 8 is formed by a dye or type of pigments which has been released, i.e. approved, for use in cosmetics, food and/or drugs. Suitable dyes absorb light in the near infrared (NIR) wavelength range. It has been found that dyes having the following Color Index (CI) are particularly well suited:

| CI (#) | example dye name | supplier |
|---|---|---|
| 74220 | Cu Pc tetrasulfonato | Sigma |
| 74160 | Cosmenyl Blue A2R/A4R | Clariant |
| 77266 | Cosmenyl Black R | Clariant |
| 74260 | Cosmenyl Green GG | Clariant |
| 61570 | Acid Green 25 | Sigma |
| 61570 | Sanolin Green GL | Clariant |
| 75815 | Chlorophyllin | Warner Jenkinson |
| 10020 | Vert BW7002 | Sensient |

The Color Index (CI) is the generally used reference number for dyes and pigments which allows identification of the product. Products having the same CI may be sold under different names and by different suppliers.

Preferably, in order to achieve enhanced matching of the optical properties of the optically matching medium 6 to the turbid medium, two or more of the dyes or types of pigments released for use in cosmetics, food and/or drugs are combined such that the absorber component 8 comprises a combination of two or more dyes or types of pigments. It has been found that a combination of CI 74160 and CI 77266 (e.g. Cosmenyl Blue A2R/A4R and Cosmenyl Black R) and a combination of CI 74260 and CI 77266 (e.g. Cosmenyl Green GG and Cosmenyl Black R) are particularly well suited, but other combinations are also possible. Mixtures with CI 61570 (e.g. Acid Green 25 and Sanolin Green GL) are also very well suited.

Further, in order to achieve the desired reduced scattering coefficient μ′ₛ, a scattering component 9 is added to the base medium 7. Again, a scattering component 9 is used which is suitable for contact with the turbid medium. According to the present embodiment, TiO₂ is added as scattering component 9, but other materials having the required scattering properties are also possible. TiO₂ has the Color Index (CI) 77801. As the scattering component 9 a material is used which is released for cosmetics, food, and/or drugs. It has been found that, with regard to TiO₂, the following products are particularly suitable:

| Product | supplier |
|---|---|
| Hombitan FF Pharma | Sachtleben |
| AFDC | Kemira |
| Eurovit | Sensient |
| Tronox AZ | KMG |

It should be noted that the product names used in the tables and the names of the suppliers might be subject to trademark rights and are not intended to be used in a generic way but only to define the specific products supplied by the named suppliers.

Thus, according to the embodiment and modifications described, an optically matching medium with appropriate optical properties in the desired range of wavelengths is provided which can be used in direct contact with the turbid medium and which can be produced in a cost-efficient way. Since both, the absorber component 8 and the scattering component 9, are materials approved for cosmetics, food, and/or drugs, the optically matching medium 6 can be used in direct contact with the turbid medium which is particularly important for use in in vivo examination of humans or animals, such as for example in mammography applications.

Commercially available products released for cosmetics, food, and/or drugs are used as absorber component 8 and scattering component 9. Thus, the optically matching medium 6 can be generated in a cost-efficient way.

Since the optically matching medium 6 is generated by adding one or more commercially available dyes or types of pigments as an absorber component 8, an optically matching medium 6 having the desired optical properties can be generated depending on the specific wavelength used and on the specific type of turbid medium. Further, the required optically matching medium 6 can be designed in a short time and cost-efficiently.

1. Optically matching medium (6) for use in a device for examining turbid media, the optically matching medium (6) comprising an absorber component (8) providing the optically matching medium with predetermined absorption properties,
wherein the absorber component (8) is selected such that the optical properties of the optically matching medium (6), with respect to electromagnetic radiation having a wavelength between 650 nm and 900 nm, substantially match the optical properties of a turbid medium (1) to be examined, and
wherein the absorber component (8) comprises a dye or pigment approved for use in cosmetics, food, and/or drugs.

2. Optically matching medium (6) according to claim 1, wherein the absorber component (8) comprises a combination of at least two dyes or types of pigments approved for use in cosmetics, food, and/or drugs.

3. Optically matching medium (6) according to claim 1, wherein the absorber component (8) comprises a dye or a combination of dyes of the group having the Color Index (CI): 74220; 74160; 77266; 74260; 61570; 75815; and 10020.

4. Optically matching medium (6) according to claim 1, wherein the dye or type of pigments is selected such that the optical properties of the optically matching medium match the optical properties of the turbid medium (1) with respect to electromagnetic radiation having a wavelength between 700 nm and 800 nm, more preferably in a range around 730 nm.

5. Optically matching medium (6) according to claim 1, wherein the optically matching medium further comprises a scattering component (9) which provides predetermined scattering properties to the optically matching medium.

6. Optically matching medium according to claim 5, wherein the scattering component (9) comprises titanium dioxide.

7. Optically matching medium (6) according to claim 1, wherein the optically matching medium is adapted to be placed between a turbid medium (1) to be examined and a holder (2) being a part of the device for examining turbid media which is structured to receive the turbid medium to be examined.

8. Optically matching medium (6) according to claim 1, wherein the optically matching medium is adapted for use in a device for optical mammography, in particular for use in a device for Diffuse Optical Tomography (DOT), and the optical properties of the optically matching medium are provided to substantially match the optical properties of a female breast as a turbid medium (1).

9. A method for obtaining an optically matching medium (6) for use in a device for examining turbid media, the method comprising the steps:
selecting at least one appropriate dye or type of pigments based on the optical properties of a turbid medium to be examined;
generating the optically matching medium by adding the at least one selected dye or type of pigments as an absorber component (8) to a base medium (7);
wherein the absorber component (8) is selected such that, after adding the absorber component (8) to the base fluid (7), the optical properties of the optically matching medium (6) substantially match the optical properties of a turbid medium to be examined with respect to electromagnetic radiation having a wavelength between 650 nm and 900 nm; and
wherein, in the step of selecting at least one dye or type of pigments, a dye or type of pigments is selected which is approved for use in cosmetics, foods and/or drugs.

10. The method according to claim 9, wherein the selected dye or combination of dyes is contained in the group having the Color Index (CI): 74220; 74160; 77266; 74260; 61570; 75815; and 10020.

11. Device for examining turbid media, the device comprising:
a holder (2) for receiving a turbid medium (1) to be examined;
at least one light source optically connected to the holder (2) for irradiating the interior of the holder (2);
at least one detector optically connected to the holder (2) for detecting light emanating from the interior of the holder (2); and
an optically matching medium (6) according to claim 1.

12. The device for examining turbid media according to claim 11, wherein the device is a medical image acquisition device.

13. Use of an optically matching medium (6) in a device for examining turbid media; the device for examining turbid media comprising:
a holder (2) for receiving a turbid medium (1) to be examined;
at least one light source optically connected to the holder (2) for irradiating the interior of the holder (2); and
at least one detector optically connected to the holder (2) for detecting light emanating from the interior of the holder (2);
wherein the optically matching medium (6) is an optically matching medium according to claim 1 which is arranged between the holder (2) and the turbid medium (1).
